(19) United States
(12) Patent Application Publication
(10) Pub. No.: US 2009/0137842 A1
(43) Pub. Date: May 28, 2009

(54) PREGABALIN -4-ELIMINATE, PREGABALIN 5-ELIMINATE, THEIR USE AS REFERENCE MARKER AND STANDARD, AND METHOD TO PRODUCE PREGABALIN CONTAINING LOW LEVELS THEREOF

(76) Inventors: Yuri VOLLERNER, Lod (IL); Yanai Golub, Maitar (IL); Lilach Hedvati, Doar Na Hefer (IL); Yuriy Raizi, Natanya (IL); Mirit Leibovich, Bear-Sheva (IL); Amihai Eisenstadt, Petach-Tikva (IL); Rahamin Aminov, Natanya (IL)

Correspondence Address: KENYON & KENYON LLP, ONE BROADWAY, NEW YORK, NY, 10004, US

(21) Appl. No.: 12/245,432

(22) Filed: Oct. 3, 2008

Related U.S. Application Data
(60) Provisional application No. 60/977,237, filed on Oct. 3, 2007.
(60) Provisional application No. 60/987,595, filed on Nov. 13, 2007.
(60) Provisional application No. 61/028,686, filed on Feb. 14, 2008.

(51) Int. Cl. C07C 209/00; G01N 33/00

(52) U.S. Cl. 562/553; 436/106

(57) ABSTRACT

The present invention provides 3-(aminomethyl)-5-methylhex-4-enoic acid (Pregabalin-4-eliminate or PRG-4E) and 3-(aminomethyl)-5-methylhex-5-enoic acid (Pregabalin-5-eliminate or PRG-5E), and their uses as reference markers and standards for determining the purity of Pregabalin. The invention also provides a method to produce Pregabalin containing low levels of these impurities.

### RELATED APPLICATIONS

This application claims the benefit of U.S. provisional application Nos. 60/977,237, filed Oct. 3, 2007; 60/987,595, filed Nov. 13, 2007; and 61/028,686, filed Feb. 14, 2008; each of which is incorporated by reference herein in its entirety.

### FIELD OF THE INVENTION

The present invention relates to 3-(aminomethyl)-5-methylhex-4-enoic acid (Pregabalin-4-eliminate or PRG-4E) and 3-(aminomethyl)-5-methylhex-5-enoic acid (Pregabalin-5-eliminate or PRG-5E), and their uses as reference markers and standards when determining the purity of Pregabalin. The invention also relates to a method to produce Pregabalin containing low levels of these impurities.

### BACKGROUND OF THE INVENTION

(S)-Pregabalin, (S)-(+)-3-(aminomethyl)-5-methylhexanoic acid, a compound having the chemical structure,

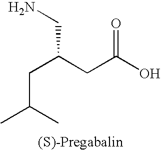

is a γ-amino butyric acid or (S)-3-isobutyl (GABA) analogue. (S)-Pregabalin has been found to activate GAD (L-glutanic acid decarboxylase). (S)-Pregabalin has a dose dependent protective effect on-seizure, and is a CNS-active compound. (S)-Pregabalin is useful in anticonvulsant therapy, due to its activation of GAD, promoting the production of GABA, one of the brain's major inhibitory neurotransmitters, which is released at 30 percent of the brains synapses. (S)-Pregabalin has analgesic, anticonvulsant, and anxiolytic activity. (S)-Pregabalin is marketed under the name LYRICAS by Pfizer, Inc., in tablets of 25, 50, 75, 150, 200, and 300 mg doses.

The preparation of (S)-Pregabalin from 3-isobutylglutaric acid is disclosed in DRUGS OF THE FUTURE, 24 (8), 862-870 (1999), and in U.S. Pat. No. 5,616,793, and is described by the following Scheme:

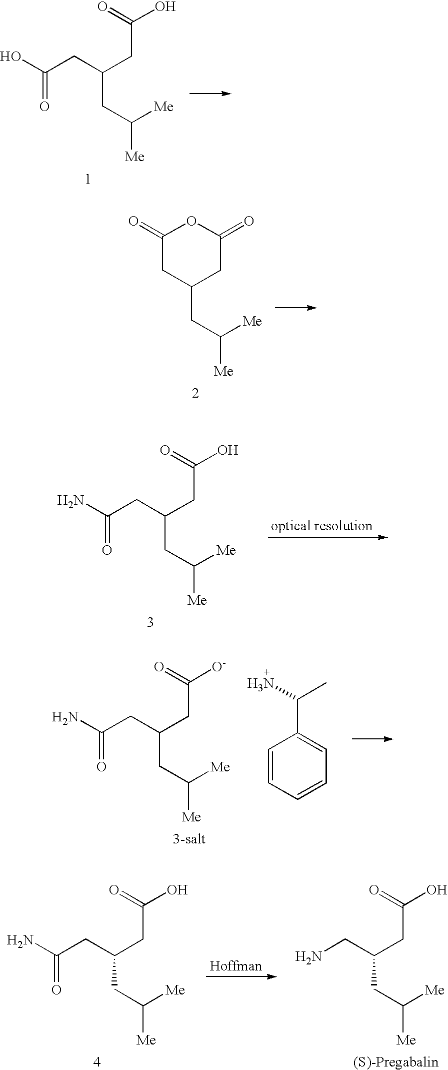

Accordingly, 3-isobutylglutaric acid, compound 1, is converted into the corresponding anhydride, compound 2, by treatment with acetic anhydride. The reaction of the anhydride with NH4OH produces the glutaric acid mono-amide, compound 3, which is resolved with (R)-1-phenylethylamine, yielding the (R)-phenylethylamine salt of (R)-3-(carbamoylmethyl)-5-methylhexanoic acid, compound 3-salt. Combining the salt with an acid liberates the R enantiomer, compound 4. Finally, Hoffmann degradation with Br₂/NaOH provides (S)-Pregabalin.

Impurities in (S)-Pregabalin or in any active pharmaceutical ingredient (API) are undesirable and, in extreme cases, might even be harmful to a patient being treated with a dosage form containing the API.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during processing of an API, such as (S)-Pregabalin, it must be analyzed for purity, typically, by HPLC or TLC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, is as safe as possible for clinical use. As discussed above, in the United States, the Food and Drug Administration guidelines recommend that the amounts of some impurities be limited to less than 0.1 percent.

Generally, side products, by-products, and adjunct reagents (collectively “impurities”) are identified spectroscopically and/or with another physical method, and then associated with a peak position, such as that in a chromatogram, or a spot on a TLC plate. (Strobel p. 953, Strobel, H. A.; Heineman, W. R., Chemical Instrumentation: A Systematic Approach, 3rd ed. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time.”

As is known by those skilled in the art, the management of process impurities is greatly enhanced by understanding their chemical structures and synthetic pathways, and by identifying the parameters that influence the amount of impurities in the final product.

Thus, there is a need in the art for managing impurities in Pregabalin and (S)-Pregabalin, thus developing a method for producing Pregabalin and (S)-Pregabalin free of various impurities.

### SUMMARY OF THE INVENTION

In one embodiment, the invention encompasses 3-(aminomethyl)-5-methylhex-4-enoic acid (Pregabalin-4-eliminate or PRG-4E) of the following formula:

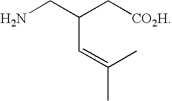

In another embodiment, the invention encompasses 3-(aminomethyl)-5-methylhex-5-enoic acid (Pregabalin-5-eliminate or PRG-5E) of the following formula:

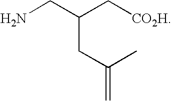

In yet another embodiment, the invention encompasses a process of determining the presence of an impurity in Pregabalin by a process comprising carrying out HPLC or TLC with the impurity as a reference marker, wherein the impurity is either PRG-4E or PRG-5E.

In one embodiment, the present invention encompasses a process of determining the amount of an impurity in Pregabalin by a process comprising carrying out HPLC with the impurity as a reference standard, wherein the impurity is either PRG-4E or PRG-5E.

In another embodiment the present invention encompasses a production scale process for preparing Pregabalin, comprising: a) reacting while stirring at a rate of about 200 rpm to about 400 rpm 3-carbamoylmethyl-5-methyl hexanoic acid (CMH), molecular halogen and about 5 to about 6 mole equivalent of a base selected from a group consisting of: alkoxide, alkali hydroxide and mixtures thereof, per mole equivalent of CMH to obtain PRG, b) extracting PRG with a C₄-₈ alcohol and a mineral acid to obtain an alcoholic phase; and c) combining the alcoholic phase with an organic base to obtain a precipitate of PRG; wherein the extraction in step b) can be a batch extraction or a multi stage extraction process. Preferably, the obtained pregabalin contains PRG-4E, PRG-5E or mixtures thereof in an amount of about 0.2% area to the detection limit of PRG-4E, PRG-5E or mixtures in an HPLC method.

### DETAILED DESCRIPTION OF THE INVENTION

As used herein, unless specified otherwise, the term “Pregabalin” refers to either the S-enantiomer of Pregabalin ((S)-Pregabalin) or to Pregabalin racemate of the following formulas:

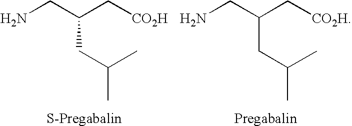

As used herein, unless specified otherwise, the term “3-(carbamoylmethyl)-5-methylhexanoic acid” or “CMH” refers to either the R enantiomer of 3-(carbamoylmethyl)-5-methylhexanoic acid or CMH ((R)—CMH) or to the CMH racemate of the following formulas:

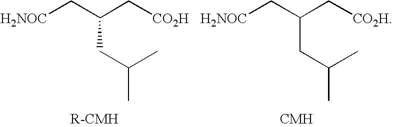

A skilled in the art would appreciate that S-Pregabalin can be prepared either from R—CMH or from CMH racemate, followed by optical resolution.

As used herein, the term “detection limit” in reference to 3-(aminomethyl)-5-methylhex-4-enoic acid (Pregabalin-4-eliminate or PRG-4E) and 3-(aminomethyl)-5-methylhex-5-enoic acid (Pregabalin-5-eliminate or PRG-5E) corresponds to the lowest level of PRG-4E or of PRG-5E that can be detected by an HPLC method. Preferably, the detection limit of the method of the present invention is 0.01% area by HPLC.

As used herein, the term “production scale” in reference to the method for producing Pregabalin corresponds to the preparation of Pregabalin from at least about 200 grams of CMH.

The present invention relates to two structurally related compounds, 3-(aminomethyl)-5-methylhex-4-enoic acid (Pregabalin-4-eliminate or PRG-4E) and 3-(aminomethyl)-5-methylhex-5-enoic acid (Pregabalin-5-eliminate or PRG-5E), methods of preparing them and isolating them, and their uses as reference markers and standards for determining their presence and amount in PRG. These compounds may be present as impurities in Pregabalin (“PRG”), and can be produced in the reaction to prepare PRG from CMH. In this reaction (the Hoffman degradation) the formed PRG reacts with the base and the molecular halogen leading to the formation of PRG-4E and/or PRG-5E in situ. The invention also relates to a method for producing PRG containing low levels of these compounds.

In one embodiment, the invention encompasses 3-(aminomethyl)-5-methylhex-4-enoic acid (Pregabalin-4-eliminate or PRG-4E) of the following formula:

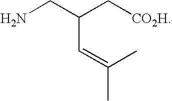

Preferably, PRG-4E is provided in an isolated form, more preferably, in a solid form, most preferably, in crystalline form. As used herein, the term “isolated” in reference to PRG-4E corresponds to PRG-4E that is physically separated from the reaction mixture. For example, the separation can be done by column chromatography on Silica gel.

More preferably, the PRG-4E is separated from PRG providing a composition of PRG-4E containing less than about 50%, more preferably, less than about 40% area by HPLC of PRG. Most preferably, the provided composition consists essentially of PRG-4E, wherein PRG is present in an amount of less than about 50%, more preferably, less than about 40% area by HPLC.

PRG-4E can be characterized by at least one of the data selected from the group consisting of: ¹H—NMR (D₂O) spectrum having peaks at about: 1.61, 1.68, 2.16, 2.88 and 4.85 ppm; ¹³C—NMR (D₂O) spectrum having peaks at about: 17.21, 24.77, 34.12, 40.03, 43.19, 122.01, 138.09, and 180.01 ppm, and mass spectra spectrum having MH⁺ peak at about 158.1 g/mole.

In one embodiment, the invention encompasses 3-(aminomethyl)-5-methylhex-5-enoic acid (Pregabalin-5-eliminate or PRG-5E) of the following formula:

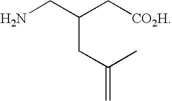

Preferably, PRG-5E is provided in an isolated form, more preferably, in a solid form, most preferably, in crystalline form. As used herein, the term “isolated” in reference to PRG-5E corresponds to PRG-5E that is physically separated from the reaction mixture. For example, the separation can be done by a preparative HPLC system. More preferably the PRG-5E is separated from PRG and PRG-4E providing a composition of PRG-5E containing less than about 50% by area of PRG-4E, and less than about 40% by area of PRG, as measured by HPLC.

PRG-5E can be characterized by at least one of the data selected from a group consisting of: ¹H—NMR (D₂O) spectrum having peaks at: about 1.63, 1.70, 2.25, 2.27, 2.95, 4.8 and 4.9 ppm; ¹³C—NMR (D2O) spectrum having peaks at about: 24.6, 32.1, 10.8, 41.0, 43.9, 113.5, 143.9, and 181.4 ppm, and mass spectra spectrum having MH⁺ peak at about 158.1 g/mole.

Typically, the amount of PRG in PRG-4E and the amount of PRG and PRG-4E in PRG-5E is measured by the HPLC method disclosed herein.

PRG-4E and PRG-5E can be prepared by a process comprising: a) reacting PRG, a base selected from a group consisting of: alkoxide, alkali hydroxide and mixtures thereof and molecular halogen to obtain a mixture, b) reacting the mixture with a mineral acid, and c) recovering PRG-4E and PRG-5E, wherein the PRG-4E and PRG-5E in step c) can be recovered as a mixture or as separate compounds.

Typically, if recovered as a mixture, PRG-4E and PRG-5E can be separated from each other in the isolation process for example, by column chromatography.

Typically, the starting PRG can be any kind of PRG, for example, crude PRG.

The starting PRG is initially combined with water to obtain a mixture. Then, the mixture is combined with the base, providing a solution.

Preferably, the alkali hydroxide is either sodium hydroxide or potassium hydroxide. Preferably, the alcoxide is sodium methoxide or sodium ethoxide. Preferably, the base is sodium hydroxide. The base can be neat, i.e., in from of a solid, or in solution. Preferably, the solution is an aqueous solution. Preferably, the aqueous solution has a concentration of about 42% to about 50%, more preferably, about 47% weight/weight.

Typically, the reaction between PRG and the base is exothermic, thus the combination of the base and the mixture of PRG in water is done upon cooling. Preferably, the cooling is to a temperature of about 20° C. to about 5° C., more preferably, to about 10° C.

Then, the solution is combined with molecular halogen providing the mixture of step a). Preferably, the molecular halogen is added to the solution. Preferably, the molecular halogen is bromine or iodine, more preferably, bromine.

Typically, the addition of the molecular halogen to the solution is exothermic, thus the temperature of the solution is maintained by cooling it. Preferably, the temperature of the solution is at about 20° C. to about 5° C., more preferably, at about 10° C.

To aid in maintaining the temperature of the solution, the addition is done portion wise. For example, when the molecular halogen is bromine, the addition can be done drop-wise, while determining the rate of addition according to the temperature of the solution.

Typically, the mixture obtained in step a) is heated. Preferably, heating is to a temperature of about 30° C. to about 80° C., more preferably, to about 40° C. Typically, the heating is done for a time sufficient to allow the formation of the salts of PRG-4E and of PRG-5E of the following formulas:

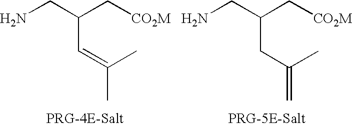

wherein, M is an alkali metal derived from the alkoxide or alkali hydroxide base, more preferably, either sodium or potassium. Preferably, heating is done for about 15 minutes to about 8 hours, more preferably, for about 15 minutes to about 2 hours.

Typically, the heated mixture is cooled and a C₄₋₈ alcohol is added prior to the addition of the mineral acid. Preferably, the C₄₋₈ alcohol is butanol, isobutanol or pentanol, more preferably, isobutanol. The addition of the mineral acid provides an ammonium salt of PRG-4E and of PRG-5E. Preferably, the mineral acid is HCl, HBr, H₃PO₄, and H₂SO₄. More preferably, the mineral acid is H₂SO₄.

Typically, the addition of the mineral acid reduces the pH to about 4 to about 2, more preferably, to about 3, providing an acidic mixture, from which both products can be recovered.

The recovery is preferably done by a process comprising reacting the acidic mixture with an organic base. Typically, the organic base neutralizes the ammonium salt to provide neutral PRG-4E and PRG-5E.

Preferably, the recovery comprises : a) heating the acidic mixture to obtain two clear phases; b) separating the organic phase from the aqueous phase; c) extrating the aqueous phase with a C₄₋₈ alcohol, d) combining this extract with the separated organic phase to obtain a new organic phase; e) cooling the new organic phase to obtain a new two phase system; f) separating the organic phase; g) cooling the separated organic phase to aid in the precipitation of inorganic salts; h) filtering these salts; i) heating the filtrate; j) adding an organic base to the filtrate to obtain a mixture; and k) cooling the mixture to obtain a suspension comprising of PRG-4E and PRG-5E.

Preferably, the organic base is selected from the group consisting of: primary amine, secondary amine, tertiary amine, aromatic amine and mixtures thereof, more preferably, either a secondary amine or tertiary amine. Preferably, the primary amine contains one C₁ to C₆ alkyl, more preferably one C₁ to C₄ alkyl. Preferably, the secondary amine contains two C₁ to C₆ alkyls, more preferably two C₁ to C₄ alkyls. Preferably, the tertiary amine contains three C₁ to C₆ alkyls, more preferably three C₁ to C₄ alkyls. Preferably, the aromatic amine is pyridine. Preferably, the secondary amine is either diisopropylamine or dipropylamine. Preferably, the tertiary amine is either tributyl amine or triethyl amine. More preferably, the organic base is tributyl amine.

The recovered mixture of PRG-4E and PRG-5E may then be further purified, thus isolating each one of the products. The purification can be done for example by column chromatography. The column chromatography allows also to separate some of the PRG-4E from PRG-5E and Pregabalin. Preferably, the column chromatography is done by using a mixture of dichloromethane:methanol:water in a ratio of 65:30:5 respectively, as a mobile phase. Further purification, i.e, isolation of PRG-5E can be done by preparative HPLC, as exemplified in Example 1.

The two compounds, PRG-4E and PRG-5E can then be used to test the purity of PRG. In one embodiment, the invention encompasses a process of determining the presence of an impurity in PRG by a process comprising carrying out HPLC or TLC with the impurity as a reference marker, wherein the impurity is either PRG-5E or PRG-4E.

Preferably, the method comprises (a) measuring by HPLC or TLC the relative retention time (referred to as RRT, or RRF, respectively) corresponding to the impurity in a reference marker sample; (b) determining by HPLC or TLC the relative retention time corresponding of the impurity in a sample comprising the impurity and PRG; and (c) determining the relative retention time of the impurity in the sample by comparing the relative retention time (RRT or RRF) of step (a) to the RRT or RRF of step (b), wherein the impurity is either PRG-4E or PRG-5E.

In another embodiment, the present invention encompasses a process of determining the amount of an impurity in PRG by a process comprising carrying out HPLC with the impurity as a reference standard, wherein the impurity is either PRG-4E or PRG-5E.

Preferably, the above process comprises: (a) measuring by HPLC the area under a peak corresponding to the impurity in a reference standard comprising a known amount of the impurity; (b) measuring by HPLC the area under a peak corresponding to impurity in a sample comprising the impurity and PRG; and (c) determining the amount of the impurity in the sample by comparing the area of step (a) to the area of step (b), wherein the impurity is either PRG-4E or PRG-5E.

Typically, the HPLC method used to determine the presence and amount of these impurities is as disclosed herein.

The invention also provides a production scale method for producing PRG containing low levels of these impurities. As used herein, the term “low levels” when referring to the amount of PRG-4E and PRG-5E in PRG corresponds to about 0.2% area to the detection limit, of PRG-4E, PRG-5E or mixtures thereof in an HPLC method.

Preferably, the amount of PRG-4E, PRG-5E or mixtures thereof in PRG is of about 0.15% area to the detection limit of PRG-4E, PRG-5E or mixtures thereof in an HPLC method, more preferably, of about 0.1% area to the detection limit of PRG-4E, PRG-5E or mixtures thereof in an HPLC method, and most preferably, of about 0.05% area to the detection limit of PRG-4E, PRG-5E or mixtures thereof in an HPLC method.

Preferably, the detection limit of the method of the present invention is 0.01% area by HPLC. Thus the present method provides PRG containing PRG-4E, PRG-5E or mixtures thereof in an amount of about 0.2% to about 0.01% area by HPLC, more preferably, of about 0.15% to about 0.01% area by HPLC, even more preferably, of about 0.1% to about 0.01% area by HPLC, most preferably, of about 0.05% to about 0.01% area by HPLC.

The production scale method comprises: a) reacting while stirring at a rate of about 200 rpm to about 400 rpm CMH, molecular halogen and about 5 to about 6 mole equivalent of a base selected from a group consisting of: alkoxide, alkali hydroxide and mixtures thereof, per mole equivalent of CMH, b) extracting PRG with a C₄₋₈ alcohol and a mineral acid to obtain an alcoholic phase; and c) combining the alcoholic phase with an organic base to precipitate PRG; wherein the extraction in step b) can be a batch extraction or a multi stage extraction process. Preferably, the amount of PRG-4E, PRG-5E or mixtures thereof in the obtained Pregabalin is of about 0.2% area to the detection limit of PRG-4E, PRG-5E or mixtures in an HPLC method.

Preferably, step a) is done as the reaction described before for preparing PRG-4E and PRG-5E is done, with the exception that the starting material is CMH and not PRG.

Preferably, the stirring rate is of about 250 rpm to about 450 rpm.

Typically, since the starting material is CMH, the heating provides the inorganic salts of PRG of the following formula

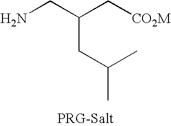

instead of the salts of PRG-4E and of PRG-5E: wherein, M is an alkali metal derived from the alkoxide or alkali hydroxide base, more preferably, either sodium or potassium.

Typically, the heated mixture is cooled prior to performing the extraction in step b). Preferably, the heated mixture is cooled to a temperature of about 40° C. to about 20° C., more preferably, to about 35° C. to about 30° C. The extraction process comprises combining the cooled mixture, a C₄₋₈ alcohol and a mineral acid. Preferably, the C₄₋₈ alcohol is isobutanol, butanol or pentanol, more preferably, isobutanol. The addition of the mineral acid provides an ammonium salt of PRG. Preferably, the mineral acid is HCl, HBr, H₃PO₄, and H₂SO₄. More preferably, the mineral acid is H₂SO₄.

Typically, the addition of the mineral acid reduces the pH, preferably to about 4 to about 2, more preferably, to about 3, providing an acidic mixture, from which PRG can be recovered after performing step c), which is a reaction with a base.

Prior to performing step c), the acidic mixture is, preferably heated to obtain a two-phase system, comprising of an alcoholic phase and an aqueous phase. Preferably, the heating is to a temperature of about 20° C. to about 40° C., more preferably to about 30° C. to about 35° C. Then, the phases are separated, and the aqueous phase can be further extracted with a C₄₋₈ alcohol, to increase the yield of PRG.

After the heating step, the alcoholic phase can be cooled to induce precipitation of inorganic salts, such as Sodium sulphate, which are removed by filtration. Preferably, the alcoholic phase is cooled to a temperature of about 15° C. to about 0° C., more preferably, to about 10° C. to about 2° C.

The alcoholic filtrate is then combined with an organic base. Typically, the organic base neutralizes the ammonium salt to provide neutral PRG. Preferably, the combination is done at about a temperature of about 10° C. to about 40° C., more preferably, of about 20° C. to about 25° C.

PRG can then be recovered for example by cooling the alcoholic filtrate, after the combination with the base to induce precipitation of PRG, and filtering it. Preferably, the cooling is to a temperature of about 2° C.

Preferably, the organic base is as described before.

### EXAMPLES

### HPLC method for determination of PRG-4E (RRT 0.5) and of PRG-5E (RRT 0.73)

HPLC

| Column &packing: | Silica C18- reverse phase (Inertsil | | |
|---|---|---|---|
| | ODS-3V 5 μm 250-4.6 mm C/N 5020-01802) | | |
| Buffer: | 0.04 M (NH4)2HPO4 adjusted to pH = | | |
| | 6.6 with H3PO4 | | |
| Eluent A: | 84% Buffer:5% Acetonitrile:11% Methanol | | |
| Eluent B: | Acetonitrile | | |
| Gradient of Eluent: | Time (min) | Eluent A (%) | Eluent B (%) |
| | 0 | 100 | 0 |
| | 6 | 100 | 0 |
| | 45 | 70 | 30 |
| | 50 | 70 | 30 |

| | |
|---|---|
| Stop time: | 50 min |
| Equilibration time: | 10 min |
| Flow: | 0.8 ml/min |
| Detector (UV): | 210 nm |
| Injection volume: | 20 μl |
| Diluent | 80% Buffer:10% Acetonitrile:10% Methanol |
| Column temperature | 25° C. |
| Autosampler | 15° C. |
| temperature | |

### PRG Standard Stock Solution

Weighed accurately about 50 mg of PRG standard into a 10 ml volumetric flask, added 8 ml of Buffer to dissolve (by sonication), added 1 ml of Acetonitrile, mixed, and made up to the volume with Methanol. Mixed well. Diluted the obtained solution 1/100 with diluent.

### 0.1% Standard Solution Preparation

Transferred 4 ml of standard stock solution into 20 ml volumetric flask and diluted up to the volume with diluent.

### Sample Solution Preparation

Weighed accurately about 100 mg of well grinded sample into a 10 ml volumetric flask, added 8 ml of Buffer to dissolve (by sonication), added 1 ml of Acetonitrile, mixed and made up to the volume with Methanol. Mixed well.

### Calculations

$$\% Imp RRT 0.50 = \frac{Area imp in smp \times Conc PRG std \times Potency PRG std ( \% )}{Conc smp \times Area PRG std \times 24.6}$$

### Where 24.6 is RRF of Impurity—RRT=0.50.

$$\% Imp RRT 0.73 = \frac{Area imp in smp \times Conc PRG std \times Potency PRG std ( \% )}{Conc smp \times Area PRG std \times 8.4}$$

### Where 8.4 is RRF of Impurity—RRT=0.73.

### EXAMPLE 1

### Preparation Of PRG-5E And PRG-4E

A reactor (10 L) was loaded with PRG-Crude (850 gr) and water (4222 gr) and. The solution was cooled to 5° C. and NaOH —47% (2153 gr) was added. Br₂ (771 gr) was added dropwise (15 min) while keeping the temperature below 10° C. The mixture was heated to 40° C. for 15 min and then cooled to 30° C. Iso-buthanol (2550 ml) and then a solution of H₂SO_{4—66}% (980 ml) were added (pH=3). The mixture was heated to 33° C., then the phases were separated, and the aqueous phase was extracted with Iso-buthanol (2125 ml). The combined organic phases were cooled to 15° C. for 2.5 h, and the phases were separated. The organic phase was cooled to 2° C. and then filtered to remove inorganic salts. The filtrate was heated to RT, and Bu₃N (1132 g) was added to the organic phase. The mixture was cooled to 0° C., and stirred for 1 h.

The solid was filtered and the cake washed with iBuOH (850 ml) to obtain of Pregabalin crude (506.4 gr, wet product) that contained 50% on area by HPLC of PRG-4-eliminate and 7 % on area by HPLC of PRG-5-eliminate.
PRG-4-eliminate was isolated by column chromatography on Silica gel in mobile phase of CH₂Cl₂—MeOH—Water (65:30:5). The fractions containing pure PRG-4-eliminate were evaporated and dried.
PRG-4-eliminate was characterized by Mass Spectra (MH⁺⁼158.1), ¹H—NMR and ¹³C—NMR (in D₂O, 400 MHz):

### ¹H—NMR:

| | |
|---|---|
| 1.61 ppm | 3H (singlet) |
| 1.68 ppm | 3H (singlet) |
| 2.16 ppm | 2H (double quartet) |
| 2.88 ppm | 2H (multiplet) |
| 4.85 ppm | 1H (doublet) |

### ¹³C—NMR:

| | | |
|---|---|---|
| 17.21 | ppm | |
| 24.77 | ppm | |
| 34.12 | ppm | |
| 40.03 | ppm | |
| 43.19 | ppm | |
| 122.01 | ppm | |
| 138.09 | ppm | |
| 180.01 | | (CO₂H) |

PRG-5-eliminate was isolated from the non-pure fractions obtained in the above column, by using the preparative HPLC system—Column Silica C18-reverse phase (Intersif 7MKN 50×259 nm), Mobile phase—H₂O/MeOH/ACN (8:1:1)+Ammonium acetate (0.04M).
PRG-5-eliminate was characterized by Mass Spectra (MH⁺=158.1), ¹H—NMR and ¹³C—NMR (in D₂O, 400 MHz):

### ¹H—NMR:

| | |
|---|---|
| 1.63 ppm | 1H (multiplet) |
| 1.70 ppm | 3H (singlet) |
| 2.25 ppm | 2H (multiplet) |
| 2.27 ppm | 2H (multiplet) |
| 2.95 ppm | 2H (multiplet) |
| 4.8 ppm | 1H (singlet) |
| 4.9 ppm | 1H (singlet) |

### ¹³C—NMR:

| | |
|---|---|
| 24.6 ppm | |
| 32.1 ppm | |
| 40.8 ppm | |
| 41.0 ppm | |
| 43.9 ppm | |
| 113.5 ppm | |
| 143.9 ppm | |
| 181.4 ppm | (CO₂H) |

The error in ¹H—NMR or ¹³C—NMR measurement is ±0.3 ppm.

### EXAMPLE 2

### Preparation Of Pure PRG-Multi Stage Extraction

A reactor was loaded with R—CMH (1.1 kg) and water (5.4 Kg). The solution was cooled to 5° C. and NaOH 47% (2.78 Kg, 5.56 eq) was added while keeping the temperature lower then 10° C. and a stirring rate of 250 rpm. Br₂ (998 gr) was added dropwise while still keeping the temperature below 10° C. The mixture was heated to 45° C. and then Iso-buthanol—saturated with water (440 ml) was added. A solution of H₂SO₄—66% (1.1-1.3 L) were added to pH=3. The obtained solution is the aqueous phase for the extraction. In second reactor the organic phase was prepared by mixing saturated isobuthanol and H₂SO₄-66% to pH 3 (3-5 L). The two streams are put into multi-stage extraction devise. The organic phase was collected and cooled to 15° C. for 1 h, seeding with Na₂SO₄ was done and the solution was cooled to 0° C. for 3 h, and then filtered to remove inorganic salts. The filtrate was heated to RT, and Bu₃N (1.46 L) was added. The mixture was cooled to 0° C., and stirred for 2 h. The solid was filtered and the cake washed with iBuOH (1.1 L). PRG- pure from PRG-4-eliminate and PRG-5-eliminate was obtained. (the impurities were not detected).

### EXAMPLE 3

### Preparation Of PRG Contaminated With PRG-4-Eliminate—Stirring Control

A reactor was loaded with R—CMH (50 gr) and water (248 gr) and. The solution was cooled to 5° C. and NaOH —47% (126.7 gr, 5.57 eq) was added. The mixture was stirred at 700 rpm and Br₂ (45 gr) was added dropwise (15 min) while keeping the temperature below 10° C. The mixture was heated to 30-35° C . Iso-buthanol (150 ml) and then a solution of H₂SO₄—66% (61 ml) were added (pH=3). The mixture temperature was kept as 35° C., then the phases were separated, and the aqueous phase was extracted with Iso-buthanol (125 ml). The combined organic phases separated. The organic phase was cooled to 0° C. and then filtered to remove inorganic salts. The filtrate was heated to RT, and Bu₃N (67 ml) was added to the organic phase. The mixture was cooled to 2° C., and stirred for 8 h. The solid was filtered and the cake washed with iBuOH (50 ml). PRG-Crude (41 gr, wet product) that contained 0.63% on area by HPLC of PRG-4-eliminate, and non detectable amount of PRG-5-eliminate was obtained.

### EXAMPLE 4

### Preparation Of PRG Contaminated With PRG-4-Eliminate And PRG-5-Eliminate—Lack of NaOH

A reactor was loaded with NaOH —47% (85 gr, 3.74 eq) and water (175 gr). The solution was cooled to below 10° C. and R—CMH (50 gr) was added. The mixture was stirred at 250 rpm, Br₂ (43.2 gr) was added dropwise (10 min) while keeping the temperature below 15° C. The mixture was heated to 650° C. for 15 min and then cooled to RT. Iso-buthanol (150 ml) and then a solution of H₂SO₄—66% (50 ml) were added (pH=0.5). The mixture was heated to 35° C., then the phases were separated, and the aqueous phase was extracted with Iso-buthanol (125 ml). The organic phase was cooled to 2° C. and then filtered to remove inorganic salts. The filtrate was heated to RT, and Bu₃N (67 ml) was added to the organic phase. The mixture was heated to 95° C. and then cooled to 2° C., and stirred for 2 h. The solid was filtered and the cake washed with iBuOH (50 ml). PRG-Crude (25.2 gr, wet product) that contained 0.33% on area by HPLC of PRG-5-eliminate and 8.11 % on area by HPLC of PRG-4-eliminate was obtained.

### EXAMPLE 5

### Preparation Of Pure PRG

A 100 L reactor was loaded with R—CMH (6 Kg) and water (248 Kg). The solution was cooled to 5° C. and NaOH −47% (15 Kg, 5.5 eq) was added. The mixture was stirred (140 rpm) and Br₂ (5.44 Kg) was added dropwise (15 min) while keeping the temperature below 10° C. The mixture was heated to 40° C. for 15 min and then cooled to RT. Iso-buthanol (18 L) and then a solution of H₂SO₄—66% (6 L) were added (pH=3). The mixture was heated to 33° C., then the phases were separated, and the aqueous phase was extracted with Iso-buthanol (15 L). The combined organic phases were cooled to 15° C. for 2.5 h, and the phases were separated. The organic phase was cooled to 2° C. and then filtered to remove inorganic salts. The filtrate was heated to RT, and Bu₃N (6.2 kg) was added to the organic phase. The mixture was cooled to 2° C., and stirred for 2 h. The solid was filtered and the cake washed with iBuOH (6 L). PRG-pure from PRG-4-eliminate and PRG-5-eliminate was obtained. (the impurities were not detected).

### EXAMPLE 6

### Preparation Of PRG-4-Eliminate

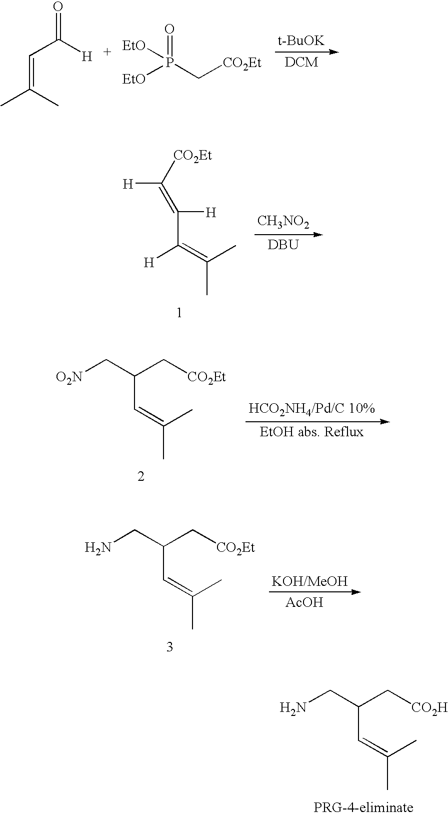

### EXAMPLE 6a

### Preparation Of Intermediate 1

A 200-ml two- necked, round bottomed flask equipped with a magnetic stirring bar, a nitrogen gas inlet, was charged with triethylphosphonoacetate (22.4 g, 0.1 M) dissolved in DCM (120 ml), and cooled in ice-bath. To the stirred mixture potassium tert-butoxide (11.2 g, 0.1 M) was added portion-wise, over a period of 10 min. The reaction mixture was stirred at the same Temp for 1 hr, after which the aldehyde (8.4 g, 0.1 M) in 15 ml DCM was added over 20 min. The reaction mixture was allowed to warm to RT, stirred overnight at RT and then diluted with water (200 ml), and extracted with DCM (50 ml). The combined extracts wash with dilute acetic acid, brine (50 ml), dried over magnesium sulfate and concentrated under reduced pressure to give a pale yellow liquid (16.6 g) which is pure intermediate 1. (Characterized By 1H—& 13-C—NMR and GCMS)

### EXAMPLE 6b

### Preparation Of Intermediate 2

In dry nitrogen filled round bottom flask, fitted with magnetic stirrer, the ester 1 (19.30 g, .125M) was dissolved in nitromethane (30 ml) and dry THF (20 ml). The solution was cooled with ice-bath, and the DBU (19 g) was added slowly over 20 min. The solution was stirred at RT for 48 hrs. The reaction mixture was poured into water (200 ml), and then ether (75 ml) and of ethylacetate (100 ml) was added. The organic phase was separated and washed with 1N HCl (100 ml), and water (100 ml), dried (MgSO4).
The solvents were evaporated and the residue was purified on Silica gel to give 7.3 g nitro compound 2 (eluted with hexane: EA 8:2 & 3:1).

### (Characterized by 1H—& 13-C—NNM, IR FAB-MS)

### EXAMPLE 6c

### Preparation Of Intermediate 3

A mixture of the nitro intermediate (2.05 g, 9.5 mmol) in EtOH (20 ml), Pd/C 10% (205 mg) and ammonium formate (0.7 g) was heated to 70° C. for 12 hrs. A second portion of catalyst Pd/C 10% (0.2 g), and ammonium formate (0.45 g) were added to the reaction mixture which was further heated to 70° C. over 24 hrs. Most of the ethanol was removed under reduced pressure, water (10 ml) was added and the pH was adjusted to 7 with few drops of ammonium hydroxide. Extraction with ethylacetate (20 ml), was followed by wash with water, brine and evaporation of the solvent under reduced pressure.
The oily residue was purified on silica-gel to yield 0.3 g of crystalline compound (eluted with acetone:hexane 2:1) which identified as the proper amino ester, 3. The product was characterized by HPLC and NMR.

### EXAMPLE 6d

### Preparation Of PRG-4-Eliminate

A 100 ml three necked round bottom flask was charged with 3 (3 g), HCl −6N (60 ml) and AcOH (2 ml), the slurry was heated to reflux for 3h. Water and excess of HCl were removed to afford an oil which washed with MTBE (2*15 ml). Water was added to the oil and the pH of the solution was adjusted with to 5.5 while using KOH, PRG-4-eliminate was precipitated. Further purification was done by dissolving the above precipitate in a minimum amount of hot water to which sufficient hot EtOH was added until crystallization appeared.

The crude product could be purified by crystallization in IPA-14% solution. characterized by HPLC and NMR.

What is claimed is:

1. 3-(aminomethyl)-5-methylhex-4-enoic acid of the following formula:

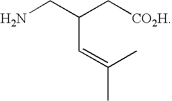

2. The compound of claim 1, wherein the compound is isolated.

3. The compound of claim 2, wherein the compound is solid.

4. The compound of claim 3, wherein the compound is crystalline.

5. The compound of claim 1 or 2, characterized by at least one of the data selected from the group consisting of: ¹H—NMR (D₂O) spectrum having peaks at: 1.61, 1.68, 2.16, 2.88 and 4.85 ppm±0.3 ppm; ¹³C—NMR (D₂O) spectrum having peaks at about: 17.21, 24.77, 34.12, 40.03, 43.19, 122.01, 138.09, and 180.01 ppm, mass spectra spectrum having MH⁺ peak at about 158.1 g/mole, and combination thereof.

6. 3-(aminomethyl)-5-methylhex-5-enoic acid of the following formula:

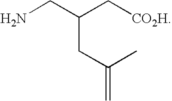

7. The compound of claim 6, wherein the compound is isolated.

8. The compound of claim 7, wherein the compound is solid.

9. The compound of claim 8, wherein the compound is crystalline.

10. The compound of claim 6 or 7 characterized by at least one of the data selected from the group consisting of: ¹H—NMR (D₂O) spectrum having peaks at about: 1.63, 1.70, 2.25, 2.27, 2.95, 4.8 and 4.9 ppm±0.3 ppm; ¹³C—NMR (D₂O) spectrum having peaks at about: 24.6, 32.1, 10.8, 41.0, 43.9, 113.5, 143.9, and 181.4 ppm, mass spectra spectrum having MH⁺ peak at about:158.1 g/mole, and combination thereof.

11. A process of determining the presence of 3-(aminomethyl)-5-methylhex-4-enoic acid (PRG-4E) or 3-(aminomethyl)-5-methylhex-5-enoic acid (PRG-5E) in a sample of Pregabalin, comprising carrying out HPLC or TLC on the sample with PRG-4E or PRG-5E as a reference marker.

12. The process of claim 11 comprising (a) measuring by HPLC or TLC the relative retention time (referred to as RRT, or RRF, respectively) corresponding to the impurity in a reference marker sample; (b) determining by HPLC or TLC the relative retention time corresponding of the impurity in a sample comprising the impurity and Pregabalin; and (c) determining the relative retention time of the impurity in the sample by comparing the relative retention time (RRT or RRF) of step (a) to the RRT or RRF of step (b), wherein the impurity is either PRG-4E or PRG-5E.

13. A process of determining the amount of 3-(aminomethyl)-5-methylhex-4-enoic acid (PRG-4E) or 3- (aminomethyl)-5-methylhex-5-enoic acid (PRG-5E) in a sample of Pregabalin comprising, carrying out HPLC on the sample with PRG-4E or PRG-5E as a reference standard.

14. The process of claim 13 comprising (a) measuring by HPLC the area under a peak corresponding to the impurity in a reference standard comprising a known amount of the impurity; (b) measuring by HPLC the area under a peak corresponding to impurity in a sample comprising the impurity and PRG; and (c) determining the amount of the impurity in the sample by comparing the area of step (a) to the area of step (b), wherein the impurity is either PRG-4E or PRG-5E.

15. A production scale process for the preparation of Pregabalin (PRG), comprising: a) reacting while stirring at a rate of about 200 rpm to about 400 rpm 3-carbamoylmethyl-5-methyl hexanoic acid (CMH), molecular halogen and about 5 to about 6 mole equivalent of a base selected from the group consisting of: alkoxide, alkali hydroxide and mixtures thereof, per mole equivalent of CMH; b) extracting PRG with a C₄₋₈ alcohol and a mineral acid to obtain an alcoholic phase; and c) combining the alcoholic phase with an organic base to obtain a precipitate of PRG; wherein the extraction in step b) can be a batch extraction or a multi stage extraction process.

16. The process of claim 15, wherein the obtained Pregabalin contains PRG-4E, PRG-5E or mixtures thereof in an amount of about 0.2% to about 0.01% area by HPLC.

17. The process of claim 15, wherein the reaction in step a) is done under a stirring rate of about 250 rpm to about 450 rpm.
